(19) United States
(12) Patent Application Publication
(10) Pub. No.: US 2003/0153832 A1
(43) Pub. Date: Aug. 14, 2003

(54) System and method for smart monitoring within a body

(76) Inventors: Jona Zumeris, Nesher (IL); Jacob Levy, Haifa (IL); Yanina Zumeris, Nesher (IL)

Correspondence Address: Eitan, Pearl, Latzer & Cohen Zedek, LLP., Suite 1001, 10 Rockefeller Plaza, New York, NY, 10020, US

(21) Appl. No.: 10/368,349

(22) Filed: Feb. 20, 2003

Related U.S. Application Data
(63) Continuation-in-part of application No. 10/348,351, filed on Jan. 22, 2003.
(60) Provisional application No. 60/349,385, filed on Jan. 22, 2002.

(51) Int. Cl. A61B 8/00

(52) U.S. Cl. 600/437

(57) ABSTRACT

A smart monitoring system for enabling remote, interactive scanning and monitoring of internal bodies. According to some embodiments of the present invention, a smart monitoring system comprises at least one Active Sensing Unit (ASU), which includes at least one vibration element for generating micro mechanical vibrations and/or receiving signals of micro mechanical vibrations, a Portable Sensing Unit (PSU), connected to the ASU (by cable or wirelessly); and at least one Central Diagnostic and Control Unit (CDCU), enabled to remotely command the PSU to control the functionality of the ASU.

### RELATED APPLICATIONS

The present U.S. patent application is a Continuation In Part of U.S. patent application Ser. No. 10/348,351 titled, “A system and method for detection of motion”, which in turn claims priority from prior U.S. provisional application No. 60/349,385, entitled “A system and method for detection of motion”.

### FIELD OF THE INVENTION

The present invention relates generally to the field of acoustic in-vivo monitoring. More particularly, the present invention relates to a system and method for smart acoustic monitoring of organs and fetuses and other objects within a body.

### BACKGROUND OF THE INVENTION

Applications using acoustic waves, in the high sonic, ultrasonic, megasonic and electromagnetic range have commonly been used for diagnostic and/or therapeutic purposes. These applications include imaging of internal body structures (e.g. organs or fetuses), limb verification, monitoring life signs of a developing fetus, determination of duration and intensity of contractions, and/or treatments requiring the application of energy to specific regions within a body.

Equipment according to the prior art is heavy, expensive, hardware and software specific, and is generally not usable outside of a clinic.

Furthermore, such monitoring devices are routinely used by health professionals only, as their operation typically requires substantial medical training. For example, operation of fetal heartbeat detection involves manually moving the device head, containing the transmitter and receiver, until the heartbeat is detected. This is because these devices typically employ ultrasonic waves that are transmitted from and received by the device in a “straight line” manner. For this reason there is a particular requirement for high precision when performing monitoring procedures, requiring expert input to perform accurate scans. These procedures are, therefore, not typically implemented by non-experts. Devices suitable for home or domestic usage are available. For example, a portable ultrasonic Doppler system is described in U.S. Pat. No. 4,413,629, and a transducer for extra-uterine monitoring of a fetal heart rate is described in U.S. Pat. No. 4,966,152. A Biophysical Fetal Monitor is described in U.S. Pat. No. 5,817,035. However, all of the above listed systems and devices are expensive, and like the professional devices, require the user to manually move portions of the device to locate the heartbeat, as these devices also operate in the fetal straight-line manner.

There is thus a recognized need for, and it would be advantageous to have, an acoustic based monitoring system and method that is relatively light weight and may be useable in remote environments. It would be desirable to have device or system which would be operable with a generic computing device, and without the need to have a medical practitioner present.

### SUMMARY

The present invention provides a smart monitoring system for enabling remote, interactive scanning, sensing and/or monitoring of internal bodies. According to some embodiments of the present invention, a smart monitoring system may include at least one Active Sensing Unit (ASU), a portable sensing unit (PSU), and a Diagnostic & Control Unit (CDCU).

The Active Sensing unit may include at least one vibrating element such as a piezo-ceramic element. The vibrating element may be a vibration transmitter, a vibration receiver or a vibration transceiver. The Portable Sensing Unit (PSU) may be connected to the ASU (by cable or wirelessly). The ASU and the PSU may be connected to at least one Diagnostic and Control Unit (CDCU) adapted to communicate and control the PSU and the functionality (e.g. scanning functions) of the ASU (e.g. determine scanning parameters of the ASU).

According to some embodiments of the present invention, an interactive monitoring method may be implemented as follows: at least one ASU may be positioned substantially in contact with a body surface area (e.g. skin) in proximity to an internal structure (e.g. organ, fetus, organ of a fetus, etc.) to be monitored. The PSU may be initiated, and the ASU may scan according to a default scanning pattern.

Scanning by the ASU may include transmitting high frequency vibrations towards internal structure(s) to be monitored and receiving vibrations therefrom. According to some embodiments of the present invention, the direction to which vibrations are transmitted may be adjusted by adjusting the amplitude of vibrations transmitted by each of a set of vibration elements. According to other embodiments of the present invention, the direction of the transmitted vibrations may be adjusted by applying a signal to one or more vibrating elements, which signal may cause the shape of the vibrating element(s) to change.

According some embodiments of the present invention, vibrations reflected from a structure may be detected by a vibration detection unit, for example a piezo-ceramic receiver or transducer. The PSU may contain circuits to detect electric signals produced by the vibration detection unit. According to some embodiments of the present invention, a frequency comparison circuit may compare the frequency of received vibrations against the frequency of transmitted vibrations. According to some embodiments of the present invention, a difference value between the frequencies of the transmitted and received signals may be used to monitor movement of an internal structure based on the Doppler shift effect.

According to some embodiments of the present invention, the direction in which vibrations are transmitted may be adjusted or scanned in a pattern, where the transmitting vibrations in each direction may produce a vibration field in the transmitting direction. A signal to noise ratio may be calculated from the received vibrations for each vibration field. The process of calculating signal to noise ratios is well known.

A direction whose vibration field results in a relatively high signal to noise ratio may be considered to produce a “good scan.” Although, initially, the transmitted vibrations may be transmitted in a scan pattern, the direction of the vibrations may be set to a specific direction once that direction is found to result in a “good scan.”

The PSU may transmit data to the CDCU. The CDCU may present the data to a user of the CDCU. If the signal is not clear enough (e.g. poor signal to noise ratio), an instruction may be issued to change the direction and/or amplitude of transmitted vibrations.

According to some embodiments of the present invention, a system and method is provided that may enable interactive and remote acoustic based monitoring.

According to some embodiments of the present invention, a system and method is provided that may enable multiple monitoring events to be conducted simultaneously.

According to some embodiments of the present invention, a system and method is provided that may enable monitoring of a mobile body, such that a patient who is active or mobile can be successfully monitored.

According to some embodiments of the present invention, an apparatus is provided for enabling remote monitoring using a Tocodynamometer (TOCO) transducer, including at least one piezo-ceramic plate, a plastic cylinder, and acoustics conducting material. The plastic cylinder may be connected to the piezo-ceramic plate. The acoustics conducting material may be located at the point of contact between the apparatus and a body.

According to some embodiments of the present invention, a method for enabling remote monitoring using a TOCO transducer is provided. The method may include placing at least one piezo-ceramic plate in a TOCO transducer, connected to a plastic cylinder in the transducer. A portable Sensing Unit may transmit electric signals to at least one piezo-ceramic plate in the TOCO transducer. The piezo-ceramic plate may be oscillated, thereby causing vibrations to be sent through a body adjacent to the TOCO transducer. The reflected vibrations from body structures in the body may be received, thereby determining movement of internal bodies being monitored.

According to some embodiments of the present invention, an apparatus is provided for reducing skin impedance during a monitoring session using, for example, ECG sensors. The apparatus may include a transducer, at least one piezo-electric element, and conductive material. The piezo-electric element may be placed within the transducer. The conductive material may cover at least part of the piezo-electric element.

According to some embodiments of the present invention, a method is provided for reducing skin impedance during a monitoring session. The method may include the transmission of electric signals in thickness mode from a Portable Sensing Unit (PSU) to at least one piezo-electric element within a transducer, thereby heating up the skin at the point of contact with the skin where the transducer is placed (by improving the circulation of blood and lymph nodes). Additionally, electric signals in longitudinal mode may be transmitted from the PSU to at least one piezo-electric element, to create differences in micro-vibrations generated by the piezo-electric element(s). The result of these vibrations may generate a pealing effect at the point of contact with the skin, thereby reducing skin impedance.

### BRIEF DESCRIPTION OF THE DRAWINGS

The subject matter regarded as the invention is particularly pointed out and distinctly claimed in the concluding portion of the specification. The invention, however, both as to organization and method of operation, together with containers, features, and advantages thereof, may best be understood by reference to the following detailed description when read with the accompanying drawings in which:

FIG. 1 is a diagram illustrating components of a smart monitoring system, according to at least one embodiment of the present invention;

FIG. 2 is a diagram illustrating components of an Active Sensing Unit (ASU), according to at least one embodiment of the present invention;

FIG. 3 is a diagram illustrating a cross section of a TOCO transducer with a piezo element, according to some embodiments of the present invention;

FIG. 4 is a diagram illustrating a vibro ECG monitor with a Piezo element, according to some embodiments of the present invention;

FIG. 5 is a graphical representation illustrating results of an implementation of a vibro ECG monitor with a Piezo element, according to some embodiments of the present invention;

FIG. 6 is a diagram illustrating a vibro ECG monitor being with a Piezo element, being implemented simultaneously with X-rays, according to some embodiments of the present invention;

FIG. 7 is a diagram illustrating a Portable Sensing Unit (PSU), according to at least one embodiment of the present invention;

FIG. 8 is a diagram illustrating local and remote Central Diagnostic and Control Units, according to at least one embodiment of the present invention;

FIG. 9 is a flowchart illustrating a method of internal body monitoring, according to at least one embodiment of the present invention;

It will be appreciated that for simplicity and clarity of these illustrations, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer apparatus, processing software, input means, or output means. Various general-purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The smart monitoring system may include at least one Active Sensing Unit (ASU), a Portable Sensing Unit (PSU), and a Central Diagnostic and Control Unit (CDCU). The system may produce and process micro-vibrations, and may use these micro-vibrations to enable intelligent, remote, interactive control over sensing fields generated by ASU(s) in internal bodies etc., hereinafter referred to as “smart monitoring”. Smart monitoring may enable non-professionals to operate the monitoring apparatus in any environment, and the monitoring may be further administered by health care professionals in remote locations, by remotely adjusting the sensors of Active Sensing Unit (ASU), which may be equipped with multiple piezo-ceramic elements.

A system and method for executing remote control of a portable scanning system is described in U.S. Pat. No. 6,454,716, by the same inventor, which is incorporated herein by reference in its entirety. The '716 invention integrates at least one piezo-ceramic transmitter and receiver, and enables improved monitoring and detection of a fetal heart beat in particular.

According to some embodiments of the present invention, the integration of piezo-ceramic elements into a number of probe types may enable remote usage of and control over micro-vibrations. Such remote usage of and control over micro-vibrations may enable manipulation of sensing fields generated by ASU(s), and therefore improved performance and control of various probes, including ultrasound, ECG, TOCO (cervical contractions), and various other probe types.

Vibrating elements, such as piezo-ceramic elements, may receive electrical signals from oscillators, following which they may produce vibrations in thickness mode, longitudinal mode and/or bending (torsion) mode etc. For example, the usage of Megahertz (MHz) frequency range thickness mode vibrations may enable detection of internal bodies being monitored. This may be achieved by converting electrical signals to mechanical waves that may be sent through the body being monitored. Furthermore, the usage of Kilohertz (KHz) frequency range longitudinal mode vibrations and/or torsion, or bending mode vibrations may enable the monitoring system to achieve Doppler-type scanning. Vibrations of piezo ceramic elements in the KHz range may cause the scanning effect of MHz range ultrasound (sensing). The vibrating in the KHz range plate may change the angle of the resulting detection waves. The usage of these various vibration modes in conjunction with piezo-ceramic elements is described in greater detail in U.S. patent application Ser. No. 10/348,351, by the same inventors, which is incorporated by reference in its entirety.

Scanning by the ASU, according to some embodiments of the present invention, may include transmitting high frequency vibrations towards internal structure(s) to be monitored and receiving vibrations therefrom. According to some embodiments of the present invention, the direction to which vibrations are transmitted may be adjusted by adjusting the scanning parameters, such as the amplitude of vibrations transmitted by each of a set of vibration elements. According to other embodiments of the present invention, the direction of the transmitted vibrations may be adjusted by applying a signal to one or more vibrating elements, which signal may cause the shape of the vibrating element(s) to change.

According some embodiments of the present invention, vibrations reflected from a structure may be detected by a vibration detection unit, for example a piezo-ceramic receiver or transducer. The PSU may contain circuits to detect electric signals produced by the vibration detection unit. According to some embodiments of the present invention, a frequency comparison circuit may compare the frequency of received vibrations against the frequency of transmitted vibrations. According to some embodiments of the present invention, a difference value between the frequencies of the transmitted and received signals may be used to monitor movement of an internal structure based on the Doppler shift effect.

According to some embodiments of the present invention, the direction in which vibrations are transmitted may be adjusted or scanned in a pattern, where the transmitting vibrations in each direction may produce a vibration field in the transmitting direction. A signal to noise ratio may be calculated from the received vibrations for each vibration field. The process of calculating signal to noise ratios is well known.

A direction whose vibration field results in a relatively high signal to noise ratio may be considered to produce a “good scan.” Although, initially, the transmitted vibrations may be transmitted in a scan pattern, the direction of the vibrations may be set to a specific direction once that direction is found to result in a “good scan.” A direction whose-vibration field results in a relatively low signal to noise ratio may be considered to produce a “weak scan.” A “weak scan” may require of a user (professional or other) using the CDCU, or the CDCU itself, to alter the scanning signals and monitor the signal results, further altering the signal parameters where necessary until a “good scan” is attained.

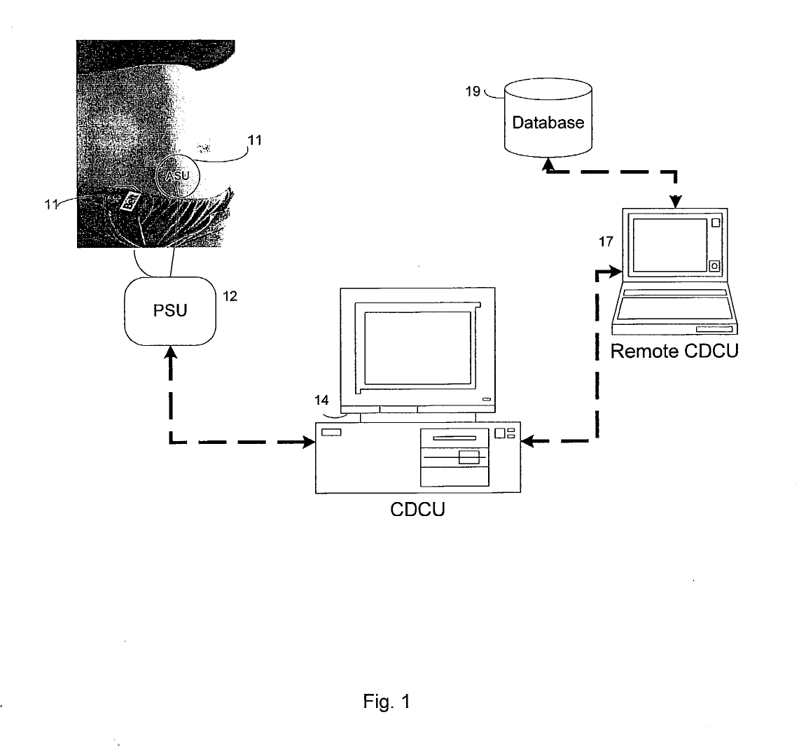

Reference is now made to FIG. 1, which illustrates the components of smart monitoring system, according to an embodiment of the present invention. As can be seen in FIG. 1, there may be provided at least one Active Sensing Unit (ASU) 11, a Portable Sensing Unit (PSU) 12, a local Central Diagnostic and Control Unit (CDCU) 14, and a remote CDCU 17. The ASU 11 may be connected (either wirelessly or by a wireline) to the PSU 12, and according to some embodiments of the present invention, the PSU 12 may be integrated into an ASU 11. PSU 12 may typically be in close proximity to the person being monitored (such as tied to a belt, or placed in a bag), so that the vibration data can be received from the ASU 11 rapidly and accurately. Vibration data from the monitoring procedure may subsequently be transferred, optionally in real time, from the PSU 12 to the CDCU 14, either wirelessly or by a wireline, using either half or full duplex. According to some embodiments of the present invention, the PSU 12 may store vibration data in a memory unit (not shown), and load the data into CDCU 14 upon accessing the CDCU 14. The local CDCU 14 may transfer the data to a remote CDCU 17, using either a wireline and/or wirelessly, optionally in real time. A care professional may use the remote CDCU 17 to communicate to and control the PSU from a remote location, optionally connecting to an external data source 19 for additional relevant data. The PSU, in turn, may communicate with and control the functionality (scanning fields etc.) of the ASU.

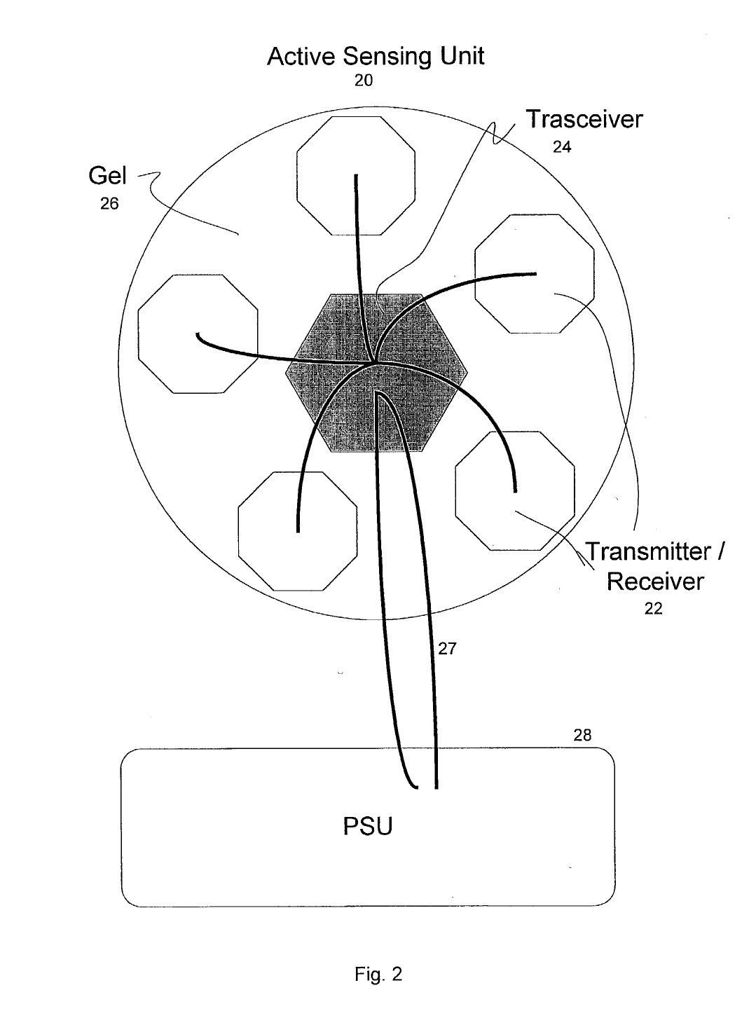

The ASU 20, as can be seen in FIG. 2, may be an interactive sensor unit (transducer). The ASU may contain a plurality of vibration elements, such as piezo elements. For example, a (central) piezo element 24 and (surrounding) piezo elements 22, or various other arrangements and forms may be used. Elements 22 and 24 may function as transmitters and/or receivers, are may hereinafter be referred to as “transceivers”, which provide vibration transmitting and/or receiving functions. For example piezo element 24 may operate as a transmitter and piezo elements 22 may operate as receivers. Alternatively, for example, piezo element 24 may operate as a receiver, and piezo elements 22 may operate as transmitters. Any other combination of element functionalities may be configured. Relevant scanning methods are detailed in U.S. patent application Ser. No. 10/348,351 by the same author titled, “A system and method for detection of motion”, which is hereby incorporated by reference in its entirety. Using the remote control functionality of a CDCU (described below), it may be possible to remotely change the acoustic signals gained from the ASU, and select the appropriate signals. Thus, when connected to an oscillator, each of the piezo-ceramic elements may be individually manipulated to oscillate synchronously over a predetermined range of voltages and frequencies, thereby transmitting energy waves over a determined angular range. Control over electric currents transmitted to the piezo-ceramic elements may therefore enable manipulation of the respective scanning fields of the various transmitters, enabling optimizing the scanning data received by the receiver, without geographically moving the ASU 20 to a different place on the body being monitored. The ASU(s) 20 may be remotely controlled by PSU 28. The piezo-ceramic elements within the transceiver units may be of any shape or size.

The smart monitoring system may include a plurality of ASU's, thereby providing various monitoring and/or sensing functions simultaneously. For example, the smart monitoring system may comprise one or more ASUs, which may include sensor devices including: uterine contraction monitors, such as Tocodynamometer (TOCO) transducers, heart activity monitors (such as electrocardiogram (ECG) recorders), heart rate monitors, arterial blood characteristic monitors (such as pulse oximetry monitors for monitoring blood oxygen level and blood pressure ), glucometers and various other non-invasive monitoring instrumentation. Such combined functionality may enable a smart monitoring system to simultaneously monitor a birthing mother's heart, glucose levels, cervix strength, as well as a baby's heartbeat etc. Such simultaneous monitoring can be further optimized due to the separate control of the various ASU's.

According to some embodiments of the present invention, the ASU may be placed or enclosed within a gel 26 container or similar other substance or compound (hereinafter referred to as “element”) that enables acoustic data transfer. In this way the container (with the ASU) may be placed adjacent to the body being monitored without requiring usage of liquid gel or other sound conducting elements. This application of the ASU in a fixed gel may provide for personalized, easy to apply, multiple usage without requiring additional application of liquid gel or alternative sound conducting elements when using the ASU.

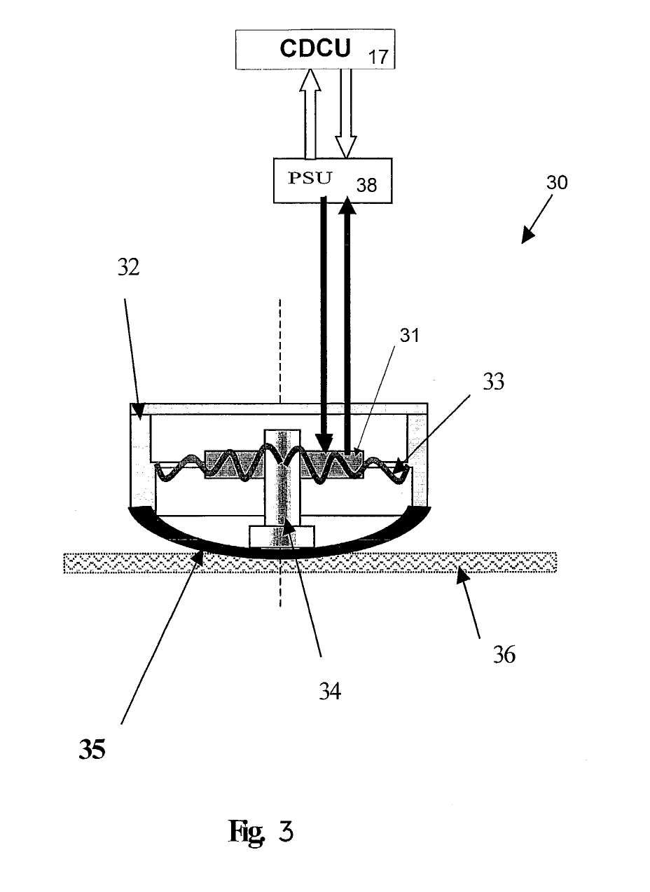

According to some embodiments of the present invention, a smart monitoring system may be incorporated into a Tocodynamometer (TOCO) transducer, or TOCO-based ASU, as can be seen according to FIG. 3, or any other type of transducer. In the case of the TOCO transducer 30, the following elements may be integrated: one or more piezo-ceramic plate 31; case 32; plastic cylinder 34, which may be connected to piezo-ceramic element 31; and acoustics conducting material 35 (comprised of rubber, gel or an alternative acoustics conducting substance for being in contact with a body and enabling mechanical waves to be transferred between the body and the piezo-ceramic elements), designed to be located at the point of contact between the TOCO-based ASU and a body being monitored. The body surface of a body being monitored is represented by 36.

After electric signals are transmitted to piezo-ceramic plate 31 at kilohertz frequencies, piezo-ceramic plate may oscillate at a determined frequency, causing vibrations 33. Internal movements from the body (for example, representing cervical contractions) may subsequently be received by the plastic cylinder 34, and may impact on the original vibrations 33. Such changes may be analyzed to determine movement of internal bodies being monitored.

TOCO transducer 30 has at least one piezo-ceramic element whose vibrations can be changed and manipulated using PSU 38. TOCO transducer vibrations may be manipulated by a local 14 and/or remote CDCU 17 to increase the accuracy or improve the signals of the monitoring session. For example, if TOCO-based ASU is being applied by a patient at a remote location, such as a patient's home environment, and is providing a poor or weak monitoring signal, a care professional at a local and/or remote CDCU may monitor the monitoring session and provide commands to remotely interact with the ASU.

In this case, for example, the care professional may alter the initial signals provided, causing changes in direction and/or multitude of the sensing fields of the ASU. Remote manipulation of the vibrating elements may thereby be enabled. In this way, a non-professional may use a sensor, such as a TOCO-based ASU, which may be controlled and optimized by a remote professional, without requiring the non-professional user to rectify or adjust the location of the ASU. Alternatively, a professional care giver may utilize various ASU based sensors, such as the TOCO-based ASU, using the additional control over field signals to attain better scan results.

Furthermore, the piezo-ceramic element in the TOCO-based ASU may enable controlled stimulation of a fetus by using vibro-acoustic waves/signals at selected megahertz and kilohertz frequencies. For example, frequencies in the range of 0,1-1 MHz may be used to stimulate a sleeping fetus.

According to some embodiments of the present invention, implementation of a smart monitoring system may be incorporated into an ECG sensor (transducer), hereinafter referred to as “Vibro ECG ASU”, as can be with reference to FIG. 4, or any other scanning mechanism or transducer. In the case of the Vibro ECG ASU 40, at least two piezo-electric elements 42 (such as piezo-ceramic element or any other element that may provide piezo effects) may be integrated into the transducer (ASU). The piezoelectric elements may be substantially thin to enable longitudinal and torsion mode vibrations to be generated. A typical thickness of such an element may be, for example, between 10-50 microns. The piezo-electric material may be covered on both sides by conductive material 43.

The piezo-electric based sensors (ASU) may enable receipt of BIO (electric) signals from the body (heart) and/or may reduce skin impedance (the disturbances between the electrode and the skin). Skin impedance is typically a problem for many monitoring devices as elements such as sweat, dirt, hair etc., may create disturbances that may interfere with signals received during monitoring sessions. In particular, typical placement of ECG sensors (electrodes) may require shaving and/or cleansing and/or rubbing of the skin where the sensor is to be applied.

According to some embodiments of the present invention, the piezo-electric element 42 may enable high quality signals to be received by the ASU from the body according to the following process:

i. various megahertz and/or kilohertz vibrations in thickness mode (47) may be generated by at least one piezo-electric element to heat up the skin at the point where the transducer (electrode) is placed. This may improve circulation of blood and lymph nodes as is known in the art, at the point of contact between the body and the sensor (electrode); and

ii. various hertz and/or kilohertz vibrations in longitudinal and/or bending and/or torsion modes (48) may be generated by at least one piezoelectric element to create micro-vibrations that may generate rubbing by the piezo-electric element(s), thereby leading to a pealing effect at the point of contact with the body;

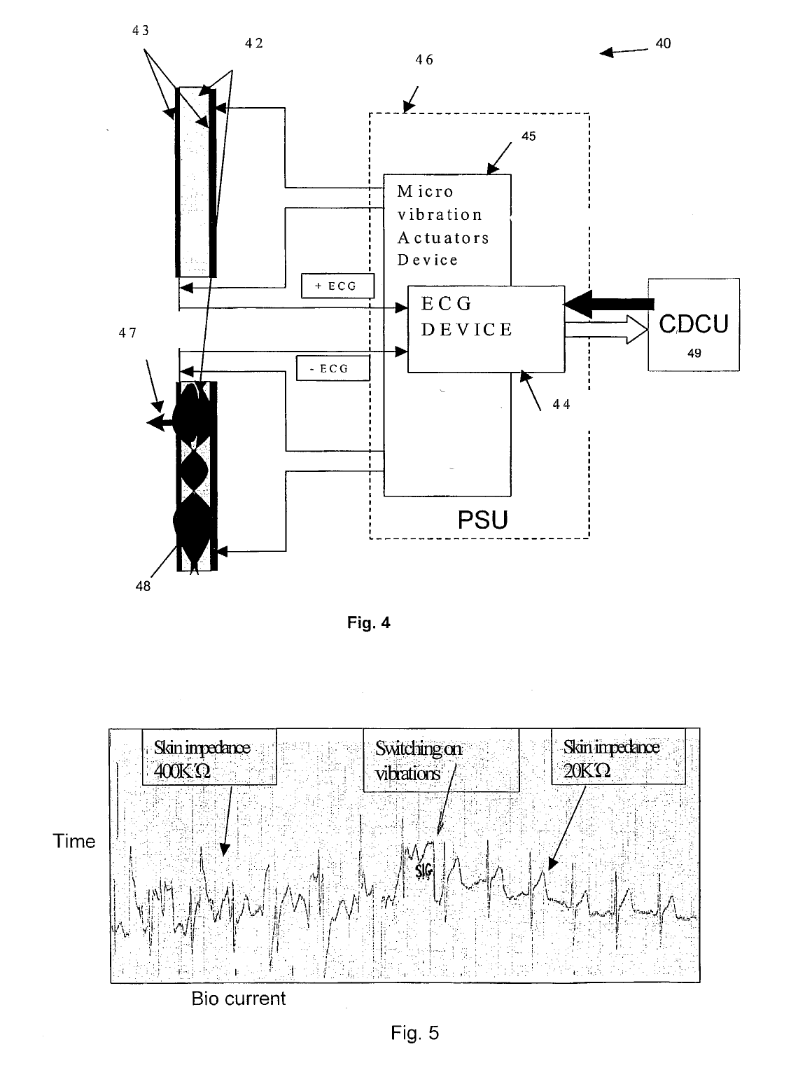

It should be noted that piezo-electric element(s) may generate thickness mode vibrations, longitudinal mode vibrations and torsion mode vibrations, either separately, together or in any combination, thereby enabling customized affects to be generated (for different body types, skin types etc.). In this way, the Vibro ECG ASU 40 may be remotely controlled by a local and/or remote CDCU, thereby manipulating the monitoring signals generated by a monitoring session. Such manipulation may be implemented by changing the power and angle of the vibrations sent into the body, thereby impacting on the quality of the monitoring signals received at the point of contact between the body and the ASU (skin impedance). As can be seen in the example provided in FIG. 5, the impedance declined from 400 Koms to 20 Koms during this procedure.

According to an embodiment, the Vibro ECG ASU 40 may be externally controlled by the PSU 46, which may include at least one micro vibration actuator driver 45 and an ECG device 44. The ECG circuitry (micro-vibration actuator) may be integrated into a PSU to enable transfer, receipt, and processing of ECG signals. The vibration actuator driver 45 may supply electrical signals to conductive material (electrode) 43 of the sensor 42, and generate vibrations of the piezo element, causing a decrease in the impedance. The ECG device 44 may measure electrical bio signals from the skin through one of the conductive layers 43. ECG device 44 may transmit the relevant data to at least one CDCU 49.

It may be necessary to electrically isolate the micro-vibration actuator and the ECG device using optical, acoustic and/or any other elements or methods known in the art. The micro-vibration actuator 40 may be connected to a CDCU wirelessly or by wireline.

Skin impedance may be substantially reduced when utilizing the vibro-effect of the Vibro ECG ASU (transducer). As can be seen with reference to FIG. 5, the point where “SIG” is marked illustrates the improved signal quality when vibrations are generated by the Vibro ECG ASU

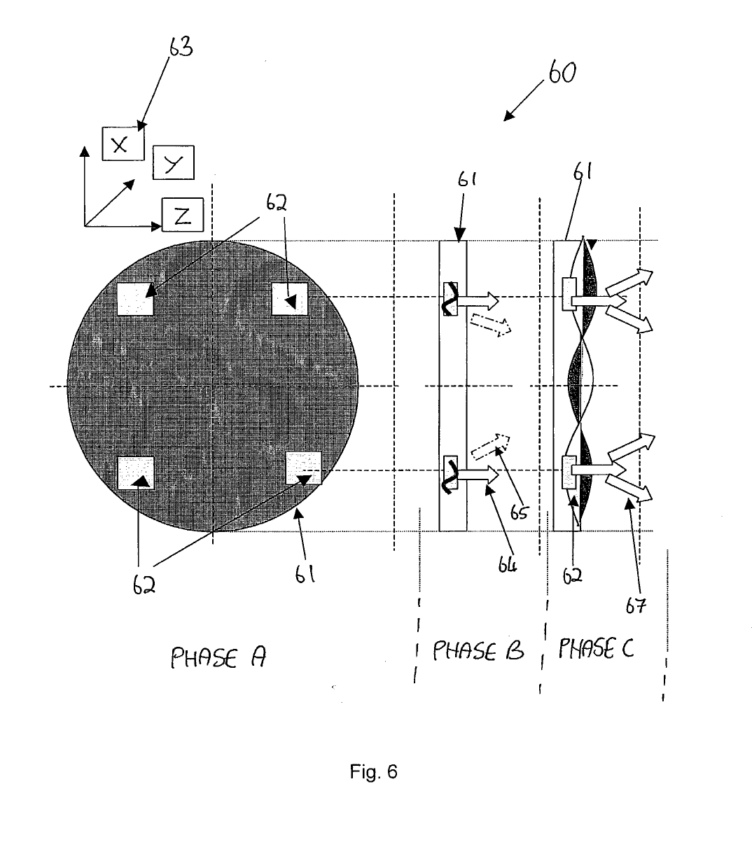

According to some embodiments of the present invention, the Vibro ECG ASU may be integrated into plastic ECG sensors, to be used simultaneously with an X-Ray procedure. As can be seen with reference to FIG. 6, a plurality of piezo-ceramic elements 62 may be integrated into a plastic ECG sensor 61. In phase A, thickness vibrations may be generated by the piezo-ceramic element, causing straight line effects illustrated 63. As a result the impedance characteristic declines only at the local point, near the piezo element. Additionally, longitudinal vibrations may be generated by the piezo-ceramic elements, causing non-straight line affects, as illustrated by 64. As a result the skin surface may be vibrated, causing decreased impedance of the sensor 61. As can be seen in Phase B, the usage of thickness and longitudinal vibrations, either separately, together or in combination, may cause a diversification of vibrations depicted as 65. In such a case the surface of the electrode 61 may begin to oscillate, causing the vibro-effect.

As can be seen in Phase C, the diversification of vibrations 67 caused by Vibro ECG ASU may result in improved scanning/sensing angles and/or a skin pealing effect. These effects may improve monitoring signals received during a monitoring session, and may alleviate procedures such as shaving the hair, cleaning, rubbing the body surface, when conducting a monitoring session.

It is to be appreciated that the skin impedance may increase over time, and it may therefore be beneficial to have the possibility of decreasing the impedance during relatively long duration monitoring sessions. Some embodiments of the present invention enable increasing and/or decreasing of impedance.

Vibro sensors may be made of piezo ceramic materials, piezo films and other materials having piezo materials as a components. In the case of piezo film (which may have a thickness of, for example, 10-50 micron) the vibrations made by the material may only be in thickness mode. For the purpose of usage of piezo films for the above-described functions, and more particularly for the purpose obtaining bending, longitudinal and torsion modes of vibrations, the electrodes of the piezo film should be split into several pieces. In such a case, by applying different voltages to different electrodes it is possible to create bending, longitudinal and torsion vibration modes.

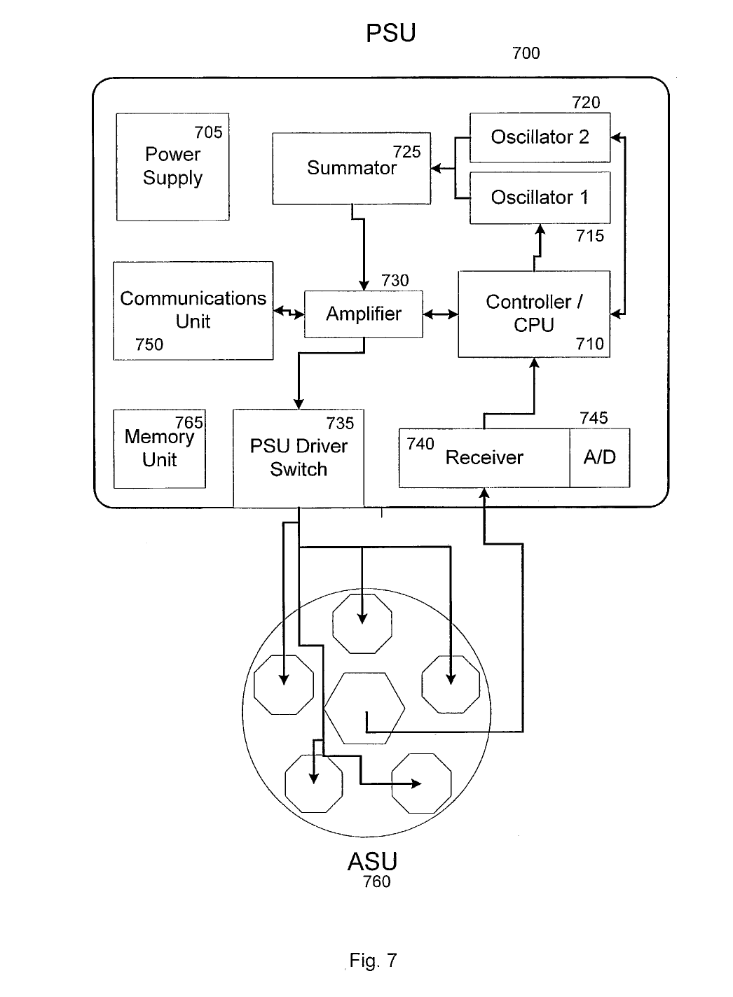

The PSU 12 is a unit that may connect to the ASU(s), either by cable(s) or wirelessly. The PSU 12 may include at least one driver circuit for each ASU that is connected to it. For simplicity sake, FIG. 7 represents the components of one driver circuit, for a PSU 700 that is connected to one ASU 760 only. In the case where a plurality of ASU's are connected to the PSU 700, there may be various additional driver circuit components required for the operation of the various ASU's. A PSU 700, as can be seen in FIG. 7 may include the following components: a power supply 705; a controller 710 (generic or dedicated logic circuit); a first oscillator 715; a second oscillator 720; a summator 725; an amplifier 730, a switch 735, a receiver 740, and a communication unit 750. The PSU 700 may also include other components. The PSU 700 may enable generation and transmission of electronic signals to the ASU(s) 760, receiving of signals from the ASU(s) 760, processing of signal data, and the communication of data to and from at least one local or remote Central Diagnostic and Control Unit (CDCU) (not shown). The communications unit 750 may enable transfer of data and receipt of data either by a cable connection or wirelessly to/from the CDCU(s). The communication unit 750 may include hardware and/or software to provide for these wire-based or wireless communications, such as RF, optical, and cellular communications. For example, the communications unit 750 may be equipped with a Bluetooth chip that enables automated receipt and transmission of relevant data to and from other Bluetooth chips in the nearby vicinity. Data may be communicated from the PSU 700 to at least one CDCU in real time.

According to a further embodiment of the present invention, the PSU 700 may contain a memory unit 765, for storing scanning data etc. Upon connection to a CDCU, the stored data in the memory unit 765 may be transferred to the CDCU and processed accordingly.

According to some embodiments of the present invention the PSU 700 may contain an analog-to-digital converter (AID) 745, for converting incoming analog signals to digitized data that may be used by the CDCU, and/or for converting incoming digital signals to analog signals that may be used by the ASU. PSU 700 may send and/or receive analog or digital signals in their original forms, without converting these signals to analog/digital form.

The Central Diagnostic and Control Unit (CDCU), which may be integrated into any computing device or environment, may provide diagnostic, control and display functions for the smart monitoring system. The CDCU may receive data from the PSU, processes the data, present the data to the user, and enable the user to interact with the PSU and/or the ASU.

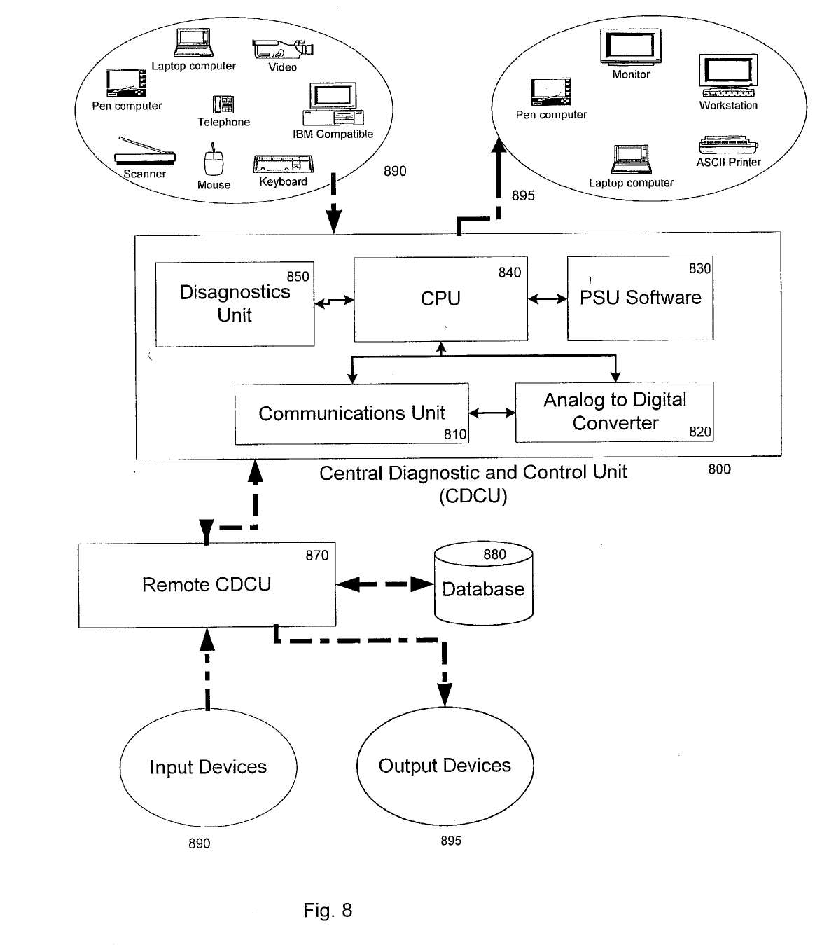

The CDCU 800, as can be seen with reference to FIG. 8, may contain a CPU (controller unit) 840 for controlling the CDCU components and functions. The CDCU 800 may further include a communications unit 810 (cable and/or wireless-based), optionally corresponding to the communications unit 750 of the PSU 700, for receiving data from and transferring data to the PSU 700. The CDCU communication unit 810 may contain hardware and/or software (including modems, chips, network devices etc.) to provide for wire-based or wireless communications (communications unit), such as cable-based, RF, optical, cellular and any other communication types. For example, the CDCU communications unit 810 may be equipped with a Bluetooth chip that enables automated receipt and transmission of relevant data to and from other Bluetooth chips in the nearby vicinity.

The CDCU 800 may additionally include an analog to digital converter unit (A/D) 820, as is commonly known in the art, for converting incoming analog signals from the PSU 700 to digitalized data that is readable to the CDCU 800, and for converting outgoing digital signals from the CDCU 800 to analog signals that are readable to the PSU 700.

In the case where PSU 700 sends and/or receives analog signals without converting these signals to/from digital form, CDCU may be equipped with at least one sound processing and presentation tool, such as a sound card and associated software, that are well known in the field. For example, SoundBlaster sound cards and analog-application software may be integrated into the CDCU to enable processing and presenting of analog sound data on a computer system. Any other relevant application hardware and/or software may be used to present the analog data to a CDCU user without converting it into digital data.

The CDCU 800 may also include PSU specific computer executable code (i.e. software 830) for receiving, transmitting, processing, analyzing, displaying, and/or communicating signal data from/to the ASU(s) and/or PSU 700. CDCU 800 may provide a GUI for enabling user interaction, and responding to user commands, thereby enabling user control of and interaction with the smart monitoring system. The PSU software 830 may include: digital imaging software for providing visual displays from the received signals; and a scanning algorithm for determining whether an ultrasonic beam from the PSU is accurate or acceptable for examination purposes. If a beam is determined to be at a non-optimal position, a PSU algorithm may provide alternative commands to one or more transmitters in an ASU to change the scanning direction or angle, and to thereby scan for more accurate signals. These commands may be provided automatically, as in the case of the scanning algorithm, or manually, where a care professional may provide commands to control the transmitter signals. In the case where different ASUs are used with a CDCU 800, CDCU 800 may have various corresponding driver circuits and/or alternative software codes for processing micro-vibration based data from the various ASUs.

The CDCU 800 may further contain peripheral hardware and/or software elements for inputting user commands and/or outputting system data. Inputting devices 890 may include: microphone, keypad, keyboard, mouse, touch screen, video camera, digitized writing pad etc. Outputting devices 895 may include: PC's, monitors, printers, speakers, PDA's etc. In addition, the CDCU 800 may contain wireless chips and/or buses for receiving data from external sources.

There may further be a remote CDCU 870, typically attended to by a medical care professional, such that the professional can remotely view monitoring results and interact with the PDU 800. Additionally, the remote CDCU 870 may be attached to a database 880 that stores patient history data, medical data, practitioner data, device data or any other relevant data, thereby supplying the attendant professional relevant data in real time. The remote CDCU 800 may provide a diagnostic unit 850 with tools for analysis of patient data, providing the patient and/or care professional with useful case data. The remote CDCU 800 may be interacted with using a variety of input devices 890 and output devices 895.

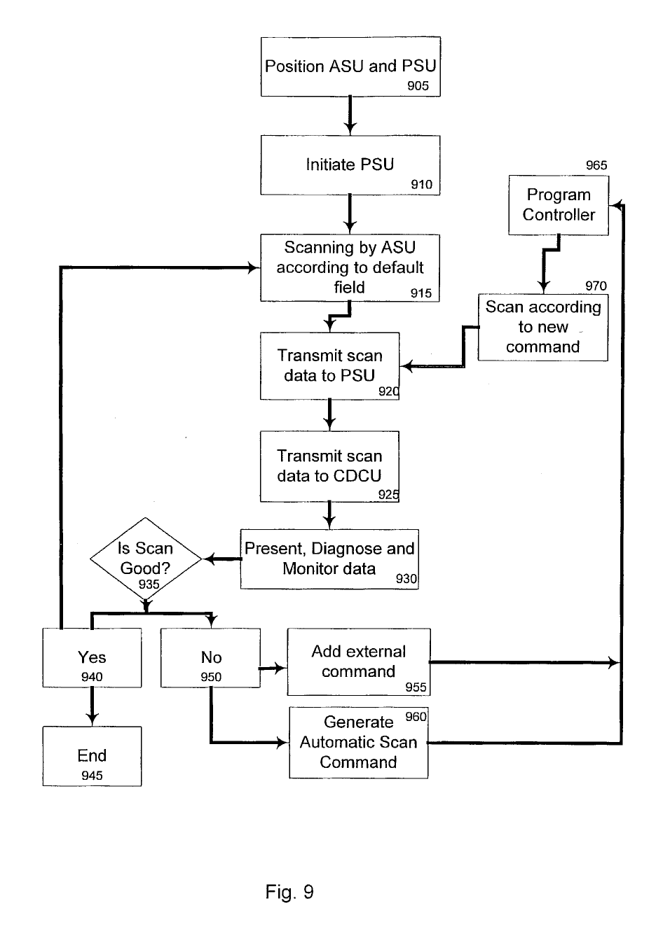

Reference is now made to FIG. 9, which is a flowchart illustrating a method of internal body monitoring, according to at least one embodiment of the present invention. As can be seen in FIG. 9, a user applies at least one ASU to a body surface 905, in the vicinity of the body structure to be monitored, and places a PSU 905 in place, such as attached to a waist belt or in a hand bag etc. The PSU is initiated (turned on) 910 and the controller administers the transmission of at least one determined acoustic signal to the ASU. An example of a method by which the Doppler pattern of transmitting and receiving acoustic signals is implemented by the PSU can be seen with reference to U.S. patent application Ser. No. 10/348,351, titled, “A system and method for detection of motion”, by the same inventor, which is incorporated by reference in its entirety. The piezo-ceramic elements in the ASU may convert the acoustic signals into micro-vibrations, sending the vibrations to the body for scanning purposes. The scanning procedure 915 may be undertaken according to a default scanning field, such as in a straight line from the probe (transducer) to the body. The vibrations may be bounced off body parts, and may subsequently be received from the scanned body by the ASU receiver(s). The piezo-ceramic elements in the ASU may convert received vibrations into electrical signals, which may subsequently be transmitted 920, either through cable or wirelessly, to the PSU communications unit. The data may subsequently be transmitted 925 to the communications unit of at least one local and/or remote CDCU, either through cable or wirelessly. In the case where there is an Analog-to-Digital Conversion Unit (A/D) in the PSU, the received signal may be converted from analog to digital format before being transferred to the CDCU. In the case where there is an AND in the CDCU, analog signals may be sent to the CDCU, where they may be converted by the A/D. A corresponding process of re-converting digital to analog signals when signals are transferred from the CDCU to the PSU and/or ASU may be executed using the relevant A/D. In the case where a memory unit is integrated into the PSU, the signal data may be stored in the memory until such a time where the PSU is connected to at least one CDCU, at which time the stored data may be transferred to a CDCU.

In the case where the PSU sends and/or receives analog signals without converting these signals to/from digital form, the CDCU may be equipped with SoundBlaster analog-application software, or any other relevant application software, to present the analog data to a CDCU user without converting it into digital data.

The CDCU may subsequently analyze, diagnose, manipulate and/or transfer the ASU data, and present the data to a user 930 (such as a care professional) on at least one output device. Either the user or the CPU of the CDCU may subsequently determine 935 whether the scan represented by the ASU data attained an acceptable and/or optimal signal. In the case where the scan is determined to be a good scan 940, the scanning session may be ended 945 or continued 915, by permitting or instructing the PSU to continue administering scanning according to the default or prior scanning dimensions or fields. In the case where the scan is determined to be below an acceptable level (not good) 950, the CDCU may be instructed to change the scanning dimensions, by instructing the PSU to alter the electric signals sent to one or more transceivers in the ASU. The instructions from the CDCU to change the scanning dimensions may be derived from external commands 955 (from a user and/or care professional) or from automated commands 960, using a scanning algorithm that automatically hones in on the target being scanned, using various scanning angles and orientations, in order to optimize the scan data received by the PSU without changing the location of the ASU on the scanned body. The PSU (program controller) may subsequently be programmed 965 with at least one new scanning command, and the new scanning command(s) may be transmitted to and implemented by at least one transmitter in the ASU. The process may then be continued 920, according to the updated scanning commands. This process may continue indefinitely throughout the scanning procedure, thereby leading to optimization of the acoustic signals.

According to some embodiments of the present invention, multiple ASU's may be provided for enabling various simultaneous smart monitoring functions.

According to some embodiments of the present invention, ASU's (such as TOCOs, ECGs, Oxymeters and glucometers) may be operated utilizing a vibro effect. This vibro effect may utilize high and low frequency oscillations (kilohertz and megahertz range) in the piezo-ceramic elements within the ASU(s), such that the biological and mechanical contact with the monitored body may be enhanced. Such an enhancement of body contact may prevent common disturbances, such as hair, sweat and dirt, skin impedance, patient movements from diminishing the results of the sensing process.

According to a further embodiment of the present invention, a command is provided by a professional user of CDCU to provide a low frequency command to stimulate a fetus. Such a command may be executed using a dedicated piezo-ceramic based transmitter in an ASU.

While preferred embodiments of the present invention have been described, so as to enable one of skill in the art to practice the present invention, the preceding description is intended to be exemplary only. It should not be used to limit the scope of the invention, which should be determined by reference to the following claims.

1. A monitoring system comprising:
at least one Active Sensing Unit (ASU), said ASU including at least one vibrating element;
a Portable Sensing Unit (PSU), connected to said ASU; and
a Central Diagnostic and Control Unit (CDCU), said CDCU enabled to command said PSU to control said ASU.

2. The system of claim 1, wherein said vibrating element is a piezo-electric element.

3. The system of claim 1, wherein said vibrating element is at least one element selected from the group consisting of a receiver, a transmitter, a transceiver, and a transducer.

4. The system of claim 2, wherein at least a portion of said piezo-electric element is a piezo-ceramic element.

5. The system of claim 2, wherein said piezo-electric element is at least one element selected from the group consisting of a piezo-electric receiver, a piezo-electric transmitter, a piezo-electric transceiver and a piezo-ceramic transducer.

6. The monitoring system of claim 1, wherein said ASU further includes a plurality of piezo-electric transmitters, and at least some of said transmitters are adapted to produce micro-vibrations.

7. The system of claim 6, wherein said micro-vibrations provide ultrasonic sensing fields.

8. The monitoring system of claim 7, wherein said CDCU is adapted to control scanning parameters of said ASU.

9. The monitoring system of claim 1, wherein said CDCU is adapted to interact remotely with said PSU.

10. The monitoring system of claim 6, wherein said micro-vibrations are in the Hertz to Megahertz range.

11. The monitoring system of claim 6, wherein said micro-vibrations have amplitudes in the nanometer to micron range.

12. The monitoring system of claim 1, wherein said ASU generates a peeling effect, thereby reducing impedance.

13. The monitoring system of claim 12, wherein said peeling effect is generated by transmitting micro-vibrations in a plurality of mode types, said mode types selected from the group consisting of thickness mode, longitudinal mode, bending mode, torsion mode and combinations of these modes.

14. The monitoring system of claim 1, wherein said ASU is placed within a substance to enable direct application to a body surface, said substance being an acoustic conducting material.

15. The monitoring system of claim 1, wherein said ASU further enables transmitting micro-vibrations to a body to stimulate a fetus.

16. The monitoring system of claim 1, wherein said ASU incorporates sensors selected from at least one of the group consisting of a uterine a contraction monitor, a heart activity monitor, a heart rate monitor, an arterial blood characteristic sensor, and a glucose level meter.

17. The monitoring system of claim 11, wherein said heart activity monitor is an ECG sensor, said ECG sensor including conductive electrodes.

18. The monitoring system of claim 16, wherein said ECG sensor enables recording of bio-signals and decreasing of impedance.

19. The monitoring system of claim 1, wherein said PSU includes at least one memory unit.

20. The monitoring system of claim 1, wherein said PSU includes at least one communication unit.

21. The monitoring system of claim 20, wherein said PSU communication unit is enabled with at least one communication circuit to communicate wirelessly with said CDCU.

22. The monitoring system of claims 19, wherein said PSU stores monitoring data in said memory unit, enabling said data to be transferred to said CDCU upon connection of said PSU to said CDCU.

23. The monitoring system of claim 20, wherein said communication unit transmits at least one data type selected from the group consisting of digital data and analog data.

24. The monitoring system of claim 20, wherein said communication unit transmits data in at least one transmission form selected from the group consisting of RF, cellular and optical.

25. The monitoring system of claim 1, wherein said CDCU includes at least one communication unit, a diagnostic unit, and computer executable code for interacting with data from said PSU.

26. The monitoring system of claim 1, wherein said CDCU further comprises an analog to digital converter.

27. The monitoring system of claim 1, wherein said CDCU is adapted to remotely determine electronic signals generated by said PSU.

28. The monitoring system of claim 1, wherein said CDCU further includes at least one sound processing and presentation tool to process and present analog data.

29. The monitoring system of claim 1, further comprising at least one remote CDCU.

30. The monitoring system of claim 1, wherein said CDCU further includes optimization code for at least one optimizing sensing function selected from at least one of the group consisting of heart rate sensing, heart activity sensing, uterine contraction sensing, arterial blood characteristic sensing, and glucose level sensing.

31. The monitoring system of claim 1, further comprising an external database.

32. An interactive monitoring method comprising:
scanning at least one internal structure according to a default scanning field, by an Active Scanning Unit (ASU), said ASU including at least one piezo-electric transmitter and at least one piezo-electric receiver;
receiving at least one scanned data signal by a Portable Sensing Unit (PSU) and transmitting said scanned data signal to at least one CDCU, by said PSU; and
interacting with said PSU in response to said scanned data received by said CDCU.

33. The method of claim 32, wherein if said scanned data signal has a relatively high signal to noise ratio, continuing with said scanning according to said default scanning field, by said ASU.

34. The method of claim 32, wherein if said scanned data has a relatively low signal to noise ratio, providing a new scan command to said PSU, by said CDCU, according to at least one operation selected from the group consisting of adding an external scan command and adding an automatic scan command.

35. The method of claim 32, wherein said scanned data signal is transmitted to a remote CDCU.

36. The method of claim 32, wherein said ASU is selected from the group consisting of a uterine contraction sensor, heart activity sensor, heart rate sensor, arterial blood characteristic sensor, and glucose level sensor.

37. The method of claim 35, wherein said new scan command is a command to generate a peeling effect to reduce impedance.

38. The method of claim 35, wherein said new scan command is a command to generate a vibration to stimulate a fetus.

39. An apparatus for monitoring using a TOCO transducer, comprising:
at least one piezo-electric plate;
a plastic cylinder, said cylinder connected to said piezo-electric plate; and
acoustics conducting material located at a point of contact of the apparatus to a body.

40. The apparatus of claim 38, wherein said piezo-electric plate is at least partially a piezo-ceramic element.

41. The apparatus of claim 39, further comprising a Portable Sensing Unit (PSU) and a Central Diagnostic and Control Unit (CDCU).

42. The apparatus of claim 39, further comprising a remote CDCU.

43. The apparatus of claim 41, wherein said CDCU is adapted to determine vibrations generated by said at least one piezo-electric plate, by commanding said PSU.

44. The apparatus of claim 42, wherein said remote CDCU is adapted to determine vibrations generated by said at least one piezoelectric plate, by remotely commanding said PSU.

45. The apparatus of claim 39, wherein said piezo-electric plate(s) generates micro-vibrations from electric signals received to said plate(s).

46. The apparatus of claim 45, wherein said micro-vibrations enable at least one function selected from the group consisting of altering the strength of resulting vibrations, and altering the angles of resulting vibrations.

47. The apparatus of claim 45, wherein micro-vibrations generated by said piezo-electric plate are generated by at least one signal selected from the group consisting of longitudinal signals, bending signals and torsion signals.

48. The apparatus of claim 45, wherein said micro-vibrations are transmitted to a fetus in said body to stimulating said fetus.

49. A method for enabling remote monitoring using a TOCO transducer, comprising:
placing at least one piezo-electric plate in a TOCO transducer,
said plate connected to a plastic cylinder in said transducer;
transmitting electric signals from a Portable Sensing Unit (PSU) to at least one piezo-electric plate in said TOCO transducer; and
sending micro-vibrations through a body adjacent to said TOCO transducer.

50. The method of claim 49, further comprising transmitting return signals resulting from said micro-vibrations to a Central Diagnostic and Control Unit (CDCU), by said PSU.

51. The method of claim 50, wherein if said return signals have a relatively low signal to noise ratio, commanding said PSU to alter said electric signals transmitted from said PSU to said ASU.

52. The method of claim 51, wherein said altered electric signals generate alternative transmission patterns, said patterns effecting vibrations according to at least one vibration change selected from the group consisting of altering the strength of the vibrations and altering the angle of the vibrations.

53. The method of claim 51, wherein a remote CDCU provides commands to alter said electric signals.

54. The method of claim 51, wherein said altered electric signals stimulate a fetus.

55. The method of claim 51, wherein said piezo-electric plate includes at least one piezo-ceramic element.

56. An apparatus for reducing impedance during a monitoring session, comprising:
i. at least one transducer;
ii. at least one piezo-electric element placed within said transducer; and
iii. conductive material covering at least part of said piezo-electric element.

57. The apparatus of claim 56, wherein said piezo-electric element includes at least one piezo film for generating micro-vibrations.

58. The apparatus of claim 56, wherein at least a portion of said piezo-electric element is a piezo-ceramic element.

59. The apparatus of claim 56, wherein said transducer is an ECG sensor.

60. The apparatus of claim 59, wherein said ECG sensor includes plastic material incorporating at least two piezo-electric plates, said plates generating variable micro-vibrations.

61. The apparatus of claim 59, wherein said ECG sensor is non-metallic, adapted for usage simultaneously with an X-ray procedure.

62. The apparatus of claim 56, wherein said variable micro-vibrations are selected from at least one of the group of vibration types consisting of thickness mode, longitudinal mode, bending mode, torsion mode and various combinations of these modes.

63. A method for reducing skin impedance during a monitoring session, comprising:
transmitting electric signals in thickness mode from a Portable Sensing Unit (PSU) to at least one piezo-electric element within a transducer; and
transmitting electric signals in longitudinal mode from said PSU to at least one piezo-electric element, to create differences in micro-vibrations generated by said piezoelectric element(s), generating a pealing effect at a point of contact between said transducer and the skin.

64. The method of claim 63, further comprising transmitting electric signals in torsion mode from said PSU to at least one piezo-electric element.

65. The method of claim 63, further comprising transmitting electric signals in at least one mode selected from the group consisting of thickness mode, longitudinal mode, torsion mode and any combination of these modes.

66. The method of claim 63, wherein said transducer is an ECG transducer.

67. The method of claim 63, wherein at least a portion of said piezo-electric element is a piezo-ceramic element.
